(19) United States
(12) Patent Application Publication
(10) Pub. No.: US 2013/0287707 A1
(43) Pub. Date: Oct. 31, 2013

(54) 6a,7,7-TRIMETHYLDECAHYDRO-1,4-METHANOCYCLOPROPA[de]NAPHTHALEN-1-OL

(71) Applicant: TAKASAGO INTERNATIONAL CORPORATION, Tokyo (JP)

(72) Inventors: Kyoko ZAIZEN, Kamakura-shi (JP); Tsuyoshi Kobayashi, Hiratsuka-shi (JP); Junko Nagano, Yokohama-shi (JP); Yoshihiro Yaguchi, Hiratsuka-shi (JP)

(73) Assignee: Takasago International Corporation, Tokyo (JP)

(21) Appl. No.: 13/866,638

(22) Filed: Apr. 19, 2013

(30) Foreign Application Priority Data
Apr. 27, 2012 (JP) 2012-103867

(51) Int. Cl. C07C 35/44; A23L 1/226; A61L 9/01; A61Q 19/00; C11B 9/00; A61K 8/34

(52) U.S. Cl. CPC C07C 35/44; C11B 9/0042; A61K 8/34; A61L 9/01; A61Q 19/00; A23L 1/22657
USPC 424/49; 568/817; 512/19; 510/104; 424/70.1; 514/772; 426/538

(57) ABSTRACT

As a novel compound which is excellent in odor quality and has an odor of a very clear and strong woody note, the invention provides a compound represented by the following formula (1):

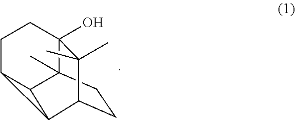

### BACKGROUND OF THE INVENTION

1. Field of the Invention

The present invention relates to a novel tetracyclic norsesquiterpene alcohol, and a method for producing a flavor/fragrance composition containing the compound. Specifically, the present invention relates to the compound having an odor of a very clear and strong woody note, and a method for producing a flavor/fragrance composition containing the compound. Furthermore, the present invention relates also to a method for producing a product containing the compound or the flavor/fragrance composition, such as a perfume, a cosmetic, a toiletry product, a food, a beverage, an oral cavity composition, or a pharmaceutical.

2. Brief Description of the Related Art

Recent diversification of perfumery, cosmetic, and toiletry products and the like has raised various expectations for flavors/fragrances used for scenting these products. With demands for flavors/fragrances capable of providing a comfortable and pleasing odor, flavors/fragrances having a highly favored and characteristic odor, and the like, there is an increasing need for flavors/fragrances which meet characteristics of individual products. At the same time, there is a demand that flavors/fragrances themselves have characteristics such as excellent odor quality, long-lasting property, and excellent compatibility with other flavors/fragrances. From the past, development and supply of a highly favored flavor/fragrance material having such characteristics to meet the demands described above have been extremely significant and important in the flavor/fragrance industries. For example, ISO E SUPER (registered trademark: manufactured by IFF), VERTOFIX (registered trademark: manufactured by IFF), and CASHMERAN (registered trademark: manufactured by IFF), and the like have been used as compounds having an odor of a woody note. However, a compound having an odor of a woody note and having high performance even in a small amount has not necessarily been sufficiently developed and supplied, and a novel compound is being sought.

U.S. Pat. No. 3,991,123 describes nortetrapatchoulol as a compound which is a tetracyclic norsesquiterpene alcohol, and which has a structure similar to that of the novel compound according to the present invention. Nortetrapatchoulol is a tetracyclic norsesquiterpene alcohol which has a three-membered ring and is obtained by chemical synthesis. U.S. Pat. No. 3,991,123 states that nortetrapatchoulol has a warm, sweet, woody, and camphor-like odor characteristic to patchouli. Meanwhile, Essential Oils; Allured Publishing, Wheaton, Ill., 1981; pp. 247-272 states that nortetrapatchoulol described in U.S. Pat. No. 3,991,123 is present in patchouli oil. However, none of these conventional art documents describes the novel compound according to the present invention.

### SUMMARY OF THE INVENTION

An object of the present invention is to provide a novel compound having an odor of a very clear and strong woody note. Another object of the present invention is to provide a method for producing a flavor/fragrance composition containing the compound having the above characteristics. Still another object of the present invention is to provide a method for producing a perfume, a cosmetic, a toiletry product, a food, a beverage, an oral cavity composition, or a pharmaceutical containing the compound or the flavor/fragrance composition.

In order to achieve the above objects, the present inventors have earnestly studied mainly various kinds of plants and various kinds of essential oils. As a result, the present inventors have found that a specific tetracyclic norsesquiterpene alcohol has an odor of a very clear and strong woody note even when alone and in a small amount. This finding has led to completion of the present invention.

Specifically, the present invention relates to a compound represented by the following formula (1):

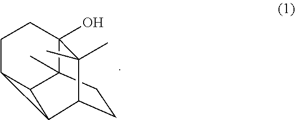

Note that the compound represented by formula (1) may be a (+)-isomer or a (−)-isomer, or may be a mixture of a (+)-isomer and a (−)-isomer, or a racemate.

In addition, the present invention also relates to a method for producing a flavor/fragrance composition, comprising mixing the compound represented by formula (1) with a flavor/fragrance component.

Moreover, the present invention also relates to a method for producing a perfume, a cosmetic, a toiletry product, a food, a beverage, an oral cavity composition, or a pharmaceutical, the method comprising: mixing the compound represented by formula (1) with a flavor/fragrance component, to thereby obtain a flavor/fragrance composition; and incorporating the obtained flavor/fragrance composition into a perfume, a cosmetic, a toiletry product, a food, a beverage, an oral cavity composition, or a pharmaceutical.

Furthermore, the present invention also relates to a method for producing a perfume, a cosmetic, a toiletry product, a food, a beverage, an oral cavity composition, or a pharmaceutical, the method comprising incorporating the compound represented by formula (1) into a perfume, a cosmetic, a toiletry product, a food, a beverage, an oral cavity composition, or a pharmaceutical.

The compound represented by formula (1) according to the present invention has an odor of a very clear and strong woody note and is excellent in diffusibility. For this reason, the compound according to the present invention can be effectively used alone as it is or in the form of a flavor/fragrance composition for various kinds of products such as a perfume, a cosmetic, a food, a beverage, a toiletry product, an oral cavity composition, or a pharmaceutical, and further can provide the above-described excellent characteristics to various products.

### DESCRIPTION OF THE PREFERRED EMBODIMENTS

Hereinafter, embodiments of the present invention will be described in detail.

<Novel Compound>

A compound according to the present invention is a novel compound represented by the following formula (1):

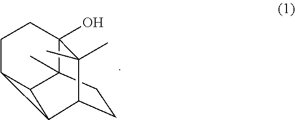

Specifically, the compound represented by formula (1) (in DESCRIPTION, also simply referred to as “the compound of the present invention”) is 6a,7,7-trimethyldecahydro-1,4-methanocyclopropa[de]naphthal en-1-ol.

The compound represented by formula (1) of the present invention has enantiomers. The compound of the present invention may be a (+)-isomer or a (−)-isomer, or may be a mixture of a (+)-isomer and a (−)-isomer or a racemate.

<<Method for Obtaining Compound of Present Invention>>

Hereinafter, a method for obtaining the compound of the present invention from patchouli oil is described. Note that operations for obtaining this compound are not particularly limited, and any approach may be employed.

(1) Leaves, stems, roots, branches, or the like of patchouli are subjected to steam distillation, and patchouli oil is obtained by collecting the oil component.

(2) The obtained patchouli oil is washed with an aqueous alkali solution, and the oil component is collected.

As the aqueous alkali solution, for example, an aqueous sodium hydroxide solution, an aqueous potassium hydroxide solution, an aqueous magnesium hydroxide solution, an aqueous calcium hydroxide solution, or the like can be used, and more preferably an aqueous sodium hydroxide solution can be used. The aqueous alkali solution can be used at a concentration in the range from 0.1 to 20% by mass, and more preferably from 0.5 to 5% by mass.

(3) The oil component obtained in (2) is fractionated by column chromatography, and a fraction having the odor is separated.

Commercially available silica gel, octadecylsilylated silica gel (ODS), or the like can be used as a separation medium for the chromatography. Preferably, silica gel is used. Examples of the eluent include water; alcohols such as methanol, ethanol, propanol, and isopropanol; halocarbons such as methylene chloride and chloroform; hydrocarbons such as pentane, hexane, benzene, and toluene; esters such as methyl acetate and ethyl acetate; acetonitrile, N,N-dimethylformamide (DMF), acetone, dimethyl sulfoxide (DMSO), tetrahydrofuran, and the like. However, the eluent is not limited thereto. In addition, one of these solvents may be used alone, or a mixture solvent thereof may be used. Of these solvents, a mixture of hexane and ethyl acetate is preferable.

(4) The fraction obtained in (3) is subjected to crystallization, and the mother liquor is obtained.

The crystallization is carried out at or below room temperature, and preferably carried out at −150° C. to 15° C., and more preferably at −90° C. to 10° C. Examples of the solvent for the crystallization include alcohols such as methanol, ethanol, propanol, and isopropanol; halocarbons such as methylene chloride and chloroform; hydrocarbons such as pentane, hexane, benzene, and toluene; esters such as methyl acetate and ethyl acetate; acetonitrile, N,N-dimethylformamide (DMF), acetone, dimethyl sulfoxide (DMSO), tetrahydrofuran, and the like. However, the solvent is not limited thereto. In addition, one of these solvents may be used alone, or a mixture solvent thereof may be used. Of these solvents, hydrocarbons are preferable, and hexane can be more preferably used.

(5) The mother liquor obtained in (4) is purified by high performance liquid chromatography to isolate the compound represented by formula (1) having an odor of a very clear and strong woody note. Examples of the column for the high performance liquid chromatography include octadecylsilylated silica gel (ODS) columns, and, for example, Inertsil (Registered Trademark) ODS-3 (manufactured by GL Sciences Inc.) is used. Examples of the eluent include water; alcohols such as methanol, ethanol, propanol, and isopropanol; halocarbons such as methylene chloride and chloroform; hydrocarbons such as pentane, hexane, benzene, and toluene; esters such as methyl acetate and ethyl acetate; acetonitrile, N,N-dimethylformamide (DMF), acetone, dimethyl sulfoxide (DMSO), tetrahydrofuran, and the like. However, the solvent is not limited thereto. In addition, one of these solvents may be used alone, or a mixture solvent thereof may be used. Of these solvents, a mixture of water and acetonitrile is preferable.

Thus, the compound represented by formula (1), which is the compound according to the present invention, can be obtained from patchouli oil. The structure of the obtained compound can be identified by ¹H-NMR and ¹³C-NMR.

Note that it is the most suitable to obtain the compound represented by formula (1) of the present invention from patchouli oil. However, besides patchouli oil, the compound can also be obtained from leaves, stems, roots, or branches of patchouli or a product (an extract, a solvent extract, or the like) obtained by processing patchouli.

The compound represented by formula (1), which is the compound according to the present invention, has an odor of a very clear and strong woody note.

### <Method for Producing Flavor/Fragrance Composition>

A method for producing a flavor/fragrance composition according to the present invention comprises mixing the compound of the present invention with a flavor/fragrance component. Here, the “flavor/fragrance component” in the present invention refers to a flavor/fragrance component other than the compound represented by formula (1) and may be a flavor/fragrance component which is contained in patchouli oil, but which is other than the compound represented by formula (1). The flavor/fragrance component is not limited to a single flavor/fragrance component, but may be a mixture of two or more flavor/fragrance components.

The method for producing a flavor/fragrance composition may include, as one mode, mixing a purified material obtained by purifying patchouli oil and containing the compound represented by formula (1) with a flavor/fragrance component.

Here, examples of the “flavor/fragrance component” used in the present invention include various synthetic flavors/fragrances, naturally occurring essential oils, synthetic essential oils, citrus oils, animal-derived flavors/fragrances, and the like. For example, a wide variety of flavor/fragrance components can be used such as those described in “Perfume and Flavor Chemicals (Aroma Chemicals) 1, 2” (Steffen Arctender (1969)), “GOUSEI KOURYOU, KAGAKU TO SYOUHIN CHISHIKI (Synthetic flavors/fragrances, Chemistry and Product Knowledge)” (issued on Mar. 6, 1996, written by Indo Motoichi, The Chemical Daily Co., Ltd.), or “Collection of Well-known Prior Arts (flavors and fragrances), Part I” (issued by Japan Patent office on Jan. 29, 1999). Of these flavor/fragrance components, typical examples include α-pinene, limonene, cis-3-hexenol, phenylethyl alcohol, styrallyl acetate, eugenol, rose oxide, linalool, benzaldehyde, muscone, MUSK T (manufactured by Takasago International Corporation, Trademark), THESARON (manufactured by Takasago International Corporation, Trademark), and the like. In addition, the use of a flavor/fragrance or the like having a cooling sensation effect such as menthol or a derivative having a menthane skeleton in combination with the compound of the present invention makes it possible to obtain a flavor/fragrance composition provided with the cooling sensation effect.

In the method for producing a flavor/fragrance composition according to the present invention, the mixing amount of the compound represented by formula (1) can be adjusted, as appropriate, between 0.001% by mass and 50% by mass relative to the total mass of the flavor/fragrance composition depending on the kinds of other “flavor/fragrance components” contained, or the purpose of use of the flavor/fragrance composition. The mixing amount of the compound represented by formula (1) is preferably 0.01% by mass to 30% by mass, and more preferably 0.1% by mass to 5% by mass relative to the total mass of the flavor/fragrance composition because of odor improvement effects such as the presence of the odor of a very clear and strong woody note, and provision of persistence, odor strength, or diffusibility.

In addition, in the method for producing a flavor/fragrance composition according to the present invention, the ratio (mass ratio) of the mixing amount of the compound of the present invention to the mixing amount of the “flavor/fragrance component” is preferably 0.1:99.9 to 30:70, and more preferably 0.3:99.7 to 15:85, from the viewpoint of odor improvement effects such as the presence of the odor of a very clear and strong woody note and provision of persistence, odor strength, or diffusibility.

The flavor/fragrance composition containing the compound represented by formula (1) has an odor of an unprecedented very clear and strong woody note, and may have a highly favored odor. In addition, when compared with flavor/fragrance compositions containing only a “flavor/fragrance component,” a flavor/fragrance composition further containing the compound represented by formula (1) may be improved in odor characteristics such as persistence, odor strength, or diffusibility.

In the method for producing a flavor/fragrance composition of the present invention, a solvent such as ethanol, dipropylene glycol (hereinafter, abbreviated as DPG in some cases), diethyl phthalate, propylene glycol, triethyl citrate, benzyl benzoate, glycerin, triacetin, or triethyl citrate may be used. In addition, if necessary, known ingredients such as an antioxidant, an ultraviolet absorber, and a coloring agent may further be mixed in the method for producing a flavor/fragrance composition according to the present invention.

### <Method for Producing Product>

The present invention also relates to a method for producing a product containing the compound represented by formula (1). In the method for producing a product of the present invention, the compound represented by formula (1) may be directly incorporated into a perfume, a cosmetic, a toiletry product, a food, a beverage, an oral cavity composition, or a pharmaceutical (in DESCRIPTION, also referred to simply as a “product according to the present invention” or a “product”). Alternatively, a flavor/fragrance composition produced by mixing the compound represented by formula (1) with the flavor/fragrance component as described above may be incorporated into a product. Moreover, the compound represented by formula (1) and the other flavor/fragrance component may be separately added to the product, and then mixed together.

The method for producing a product includes, as one mode, mixing a purified material obtained by purifying patchouli oil and containing the compound represented by formula (1) with a flavor/fragrance component, to thereby obtain a flavor/fragrance composition, and incorporating the flavor/fragrance composition into a product.

In addition, the method for producing a product may further include, as one mode, incorporating a purified material obtained by purifying patchouli oil and containing the compound represented by formula (1) into a product.

The product obtained by the present invention can be provided with a highly favored odor/flavor by the compound represented by formula (1).

<<Perfume and Cosmetic>>

Examples of a perfume and cosmetic which can be scented with the compound of the present invention or the flavor/fragrance composition containing the compound include fragrance products, basic skin care cosmetics, make-up cosmetics, hair cosmetics, sunscreen cosmetic, medicinal cosmetics, and the like.

More specifically,

examples of the fragrance products include perfumes, Eau de Parfum, Eau de Toilette, Eau de Cologne, and the like; examples of the basic skin care cosmetics include face wash creams, vanishing creams, cleansing creams, cold creams, massage creams, emulsions, lotions, cosmetic serums, beauty packs, make-up removers, and the like;

examples of the make-up cosmetics include foundations, loose face powders, pressed face powders, talcum powders, lipsticks, lip balms, cheek rouges, eyeliners, mascaras, eye shadows, eyebrow-colors, eye packs, nail enamels, enamel remover, and the like;

examples of the hair cosmetics include pomades, brilliantines, set lotions, hair sticks, hair solids, hair oils, hair treatments, hair creams, hair tonics, hair liquids, hair sprays, bandolines, hair-growing agents, hair dyes, and the like;

examples of the sunscreen cosmetic include suntan products, sunscreen products, and the like; and

examples of the medicinal cosmetics include antiperspirants, after-shaving lotions and gels, permanent wave agents, medicated soaps, medicated shampoos, medicated skin cosmetics, and the like.

<<Toiletry Product>>

Examples of the toiletry product which can be scented with the compound of the present invention or the flavor/fragrance composition containing the compound include hair-care products, soaps, body cleaning agents, bath agents, detergents, finishing softeners, cleansing agents, kitchen detergents, breaching agents, aerosol agents, air fresheners, household goods, shaving products, skin care products, repellents, tobacco products, and the like.

More specifically,

examples of the hair-care products include shampoos, rinses, two-in-one shampoos, conditioners, treatments, hair packs, and the like;

examples of the soaps include toilet soaps, bath soap, perfume soaps, transparent soaps, synthetic soaps, and the like;

examples of the body cleaning agents include body soaps, body shampoos, hand soaps, and the like;

examples of the bath agents include bath additives (bath salts, bath tablets, bath liquids, and the like), foam baths (bubble baths and the like), bath oils (bath perfumes, bath capsules, and the like), milk baths, bath jellies, bath cubes, and the like;

examples of the detergents include heavy detergents for clothes, light detergents for clothes, liquid detergents, laundry soaps, compact detergents, powder soaps, detergent powders, and the like;

examples of the finishing softeners include softeners, furniture cares, and the like;

examples of the cleaning agents include cleansers, house cleaning agents, toilet cleaners, bath cleaners, glass cleaners, mold removers, cleaners for drain pipes, and the like;

examples of the kitchen detergents include kitchen soaps, synthetic kitchen soaps, dish detergents, and the like;

examples of the breaching agents include oxidation bleaching agents (chlorine-based breaching agents, oxygen-based breaching agents, and the like), reduction bleaching agents (sulfur-based breaching agents and the like), optical breaching agents, and the like;

examples of the aerosol agents include spray-type aerosol agents, powder sprays, and the like;

examples of the air-fresheners include semi-solid, gel-, liquid-, or mist-type air-fresheners, and the like;

examples of the household goods include tissue paper, toilet paper, candles, and the like;

examples of the shaving products include shaving foams and the like;

examples of the skin care products include hand creams, body creams, body lotions, and the like.

<<Food and Beverage>>

Examples of the food and beverage which can be scented with the compound of the present invention or the flavor/fragrance composition containing the compound include beverages such as fruit juice beverages, alcoholic fruit beverages, dairy beverages, carbonated beverages, refreshing beverages, and drink preparations; cold desserts such as ice creams, sherbets, and ice pops; desserts such as jellies and crème caramels; Western confectionery products such as cakes, cookies, chocolates, and chewing gums; Japanese confectionery products such as steamed bean-jam buns, Yokan (a thick jellied dessert made of red bean paste, agar, and sugar), and Uiro (a traditional Japanese steamed cake made of rice flour and sugar); jams; candies; breads; tea beverages and other favorite beverages such as green tea, oolong tea, black tea, persimmon leaf tea, chamomile tea, Sasa veitchii tea, mulberry leaf tea, Houttuynia cordata tea, pu-erh tea, mate tea, rooibos tea, gymnema tea, guava tea, coffee, and cocoa; soups such as Japanese style soups, Western style soups, Chinese soups; flavor seasonings; various instant beverages and foods; various snack foods; and the like.

<<Oral Cavity Composition>>

Examples of the oral cavity composition which can be scented with the compound of the present invention or the flavor/fragrance composition containing the compound include dentifrices, oral cavity washes, mouth washes, troches, chewing gums, and the like.

<<Pharmaceutical>>

Examples of the pharmaceutical which can be scented with the compound of the present invention or the flavor/fragrance composition containing the compound include skin external agents such as cataplasms and ointments; internal preparations, and the like.

The mixing amount (hereinafter, also referred to as added amount) of the compound represented by formula (1) of the present invention relative to the product according to the present invention is not particularly limited, and the compound can be added at the same levels as the amounts in which common flavor/fragrance compounds are added to various kinds of perfumes or cosmetics, toiletry products, foods or beverages, oral cavity compositions, and pharmaceuticals.

A specific amount of the compound represented by formula (1) of the present invention added to a perfume or cosmetic can be preferably 0.00001 to 10% by mass, and more preferably 0.0001 to 5% by mass, relative to the total mass of the perfume or cosmetic including the compound of the present invention.

A specific amount of the compound represented by formula (1) of the present invention added to a toiletry product can be preferably 0.0001 to 5% by mass, and more preferably 0.001 to 2% by mass, relative to the total mass of the toiletry product including the compound of the present invention.

A specific amount of the compound represented by formula (1) of the present invention added to a food or beverage can be preferably 0.000001 to 1% by mass, and more preferably 0.00001 to 0.2% by mass, relative to the total mass of the food or beverage including the compound of the present invention.

A specific amount of the compound represented by formula (1) of the present invention added to an oral cavity composition can be preferably 0.000001 to 1% by mass, and more preferably 0.00001 to 0.2% by mass, relative to the total mass of the oral cavity composition including the compound of the present invention.

A specific amount of the compound represented by formula (1) of the present invention added to a pharmaceutical can be preferably 0.000001 to 1% by mass, and more preferably 0.00001 to 0.2% by mass, relative to the total mass of the pharmaceutical including the compound of the present invention.

### EXAMPLES

The present invention will be described in detail below with reference to the following non-limiting Examples and Comparative examples. It will be apparent to those skilled in the art that many changes can be made in the embodiments described without departing from the scope of the present invention.

For measuring physical properties and the like, the following equipments were used.

NMR spectrometer: AVANCE III 500 (500 MHz; manufactured by Bruker BioSpin Corporation)

Gas chromatograph-mass spectrometer: GCMS-QP2010 (manufactured by Shimadzu Corporation)
Column used: BC-WAX (50 m×0.25 mm in ID; manufactured by GL Sciences Inc.)
Oven conditions: 70° C. to 217° C., 4° C./min

Gas chromatograph-time-of-flight mass spectrometer: JMS-T100GCV (manufactured by JEOL Ltd.)
Ionization method: Field ionization method (FI)
Column used: Rxi-5 ms (30 m×0.25 mm in ID; manufactured by Restek)
Oven conditions: 50° C. (1 min) to 230° C., 4° C./min

Infrared absorption spectrometer: FT/IR-6100 (manufactured by JASCO Corporation)
Window material: barium fluoride

Polarimeter: P-1020 (manufactured by JASCO Corporation)

### Example 1

To 100 g of a 2% by mass aqueous sodium hydroxide solution, 100 g of patchouli oil M (manufactured by Takasago International Corporation) was added. The mixture was stirred at 50° C. for 30 minutes, and then the aqueous layer was separated. Thus, 100 g of an alkali-washed oil was obtained. The alkali-washed oil (30 g) was purified by normal-phase medium-pressure chromatography (silica gel column (packing material: 200 g), eluent: hexane/ethyl acetate mixture solvent (gradient=99/1 to 0/100 (volume ratio)). Thus, a fraction (3.6 g) rich in the compound represented by formula (1) was obtained. To the fraction (3.6 g), a small amount of hexane was added, and then low-temperature crystallization was carried out by using a bath cooled to approximately −80° C. with dry ice and acetone. A mother liquor (1.5 g) from which the crystals were removed was obtained. The mother liquor (1.5 g) was purified by high performance liquid chromatography using Inertsil ODS-3 as the separation column, and water/acetonitrile as the eluent (gradient condition=70/30 to 5/95 (volume ratio)). Thus, the compound represented by formula (1) was obtained (10 mg, GC purity: 94%). The isolated and purified compound had an odor of a very clear and strong woody note.

In addition, the (−)-isomer was in excess in the obtained compound of the present invention.

<Physical Data of Compound Represented by Formula (1)>

NMR Data

¹H-NMR (500 MHz, Pyridine-d5): δ 0.75-0.82 (m, 1H), 0.75-0.82 (m, 1H), 0.75-0.82 (m, 1H), 1.06-1.15 (m, 1H), 1.08 (s, 3H), 1.17 (s, 3H), 1.21 (s, 3H), 1.34 (dddd, J=12.61, 10.96, 7.66 and 1.96 Hz, 1H), 1.63 (ddd, J=13.68, 9.05 and 2.62 Hz, 1H), 1.66 (m, 1H), 1.84 (ddd, J=13.69, 11.50 and 8.86 Hz, 1H), 1.87-1.93 (m, 1H), 2.00-2.11 (m, 2H), 2.17 (ddd, J=13.01, 9.92 and 7.65 Hz, 1H), 4.70 (s, 1H)

¹³C-NMR (125 MHz, Pyridine-d5): δ 15.09 (CH), 20.63 (CH), 20.88 (CH₂), 21.18 (CH), 22.96 (CH₃), 26.58 (CH₃), 26.69 (CH₂), 27.80 (CH₂), 27.94 (CH₃), 29.18 (CH₂), 36.52 (C), 39.91 (CH), 41.28 (C), 75.60 (C—OH)

Infrared absorption spectrum (CCl₄: cm⁻¹): 3620, 2960, 1460 EI-MS data (27 eV): 206 (M+; 15), 163 (100), 178 (74), 109 (71), 123 (52), 93 (46), 79 (35), 43 (30), 67 (21), 135 (20), 145 (19), 55(17),

HRFIMS: found 206.1679, calcd. for C₁₄H₂₂O₁ (206.1671)

Specific rotation: [α]_{D}²⁰ −114.3 (c 0.83, CH₃OH)

### Example 2 and Comparative Examples 1 to 4

### Preparation of Flavor/Fragrance Compositions

Flavor/fragrance compositions were prepared according to the formulations shown in Table 1 below. The compound represented by formula (1) isolated in Example 1 was used in Example 2, whereas no compound, ISO E SUPER, VERTOFIX, and CASHMERAN were used in Comparative Examples 1 to 4, respectively. The ratio of the compound represented by formula (1) mixed in the prepared flavor/fragrance composition was approximately 0.5% by mass.

TABLE 1

| | Parts by mass | | | | |
|---|---|---|---|---|---|
| | Example | Comparative Example | | | |
| | 2 | 1 | 2 | 3 | 4 |
| Formulated flavor/ | 100 | 100.5 | 100 | 100 | 100 |
| fragrance blend A | | | | | |
| Compound of Example 1 | 0.5 | — | — | — | — |
| ISO E SUPER | — | — | 0.5 | — | — |
| (Registered Trademark) | | | | | |
| VERTOFIX | — | — | — | 0.5 | — |
| (Registered Trademark) | | | | | |
| CASHMERAN | — | — | — | — | 0.5 |
| (Registered Trademark) | | | | | |
| Total amount | 100.5 | 100.5 | 100.5 | 100.5 | 100.5 |

ISO E SUPER (Registered Trademark): 1-(1,2,3,4,5,6,7,8-octahydro-2,3,8,8-tetramethyl-2-naphthalenyl)ethanone (manufactured by IFF)
VERTOFIX (Registered Trademark): 9-acetyl-8-cedrene (manufactured by IFF)
CASHMERAN (Registered Trademark): 1,1,2,3,3-pentamethyl-6,7-dihydroindan-4(5H)-one (manufactured by IFF)

Note that the formulated flavor/fragrance blend A in Table 1 was prepared according to the formulation shown in Table 2 below.

TABLE 2
Formulated flavor/fragrance blend A

| | Parts by mass |
|---|---|
| CALONE (Registered Trademark) | 1.0 |
| CYCLOGALBANATE (Registered Trademark) | 2.0 |
| α-Damascone | 1.0 |
| Dihydromyrcenol | 200.0 |
| Ethyllinalool | 50.0 |
| HEDIONE (Registered Trademark) | 250.0 |
| HELIOBOUQUET (Registered Trademark) | 30.0 |
| cis-3-Hexenol | 1.5 |
| Lavender oil 50% BARREME | 20.0 |
| Lemon oil | 100.0 |
| γ-Methyl ionone | 20.0 |
| MUSK T (Registered Trademark) | 150.0 |
| Dipropylene glycol | Balance |
| Total amount | 900 |

CALONE: 7-methyl-2-H-1,5-benzodioxepin-3(4H)-one (manufactured by FIRMENICH)
CYCLOGALBANATE: allyl cyclohexyl glycolate (manufactured by SYMRISE)
HEDIONE: methyl dihydrojasmonate (manufactured by FIRMENICH)
HELIOBOUQUET: 2-Methyl-3-(3,4-methylenedioxyphenyl)propanal (manufactured by Takasago International Corporation)
MUSK T: ethylene brassylate (manufactured by Takasago International Corporation)

### Test Example 1

### Sensory Evaluation

Sensory evaluation was performed on the flavor/fragrance compositions obtained in Example 2 and Comparative Examples 1 to 4.

The sensory evaluation was carried out by 10 professional panelists in terms of odor quality and strength, volume, diffusibility, woody note, and amber note of their odors on 7-grade scale (7 points at the highest to 1 point at the lowest). In the evaluation, a constant amount of each composition was dropped on a smelling-strip, and the smelling-strip left at room temperature for 17 hours was tested. Table 3 shows the evaluation results, each of which is an average value of the evaluation scores of the 10 professional panelists.

TABLE 3

| Odor evaluation results | |
|---|---|
| Example | Comparative Example |

| | 2 | 1 | 2 | 3 | 4 |
|---|---|---|---|---|---|
| Odor strength | 5.8 | 4.0 | 4.1 | 4.5 | 4.3 |
| Volume | 5.4 | 4.0 | 4.0 | 4.3 | 4.4 |
| Diffusibility | 5.4 | 4.0 | 4.0 | 4.5 | 4.1 |
| Woody note | 6.0 | 4.0 | 4.1 | 4.5 | 4.0 |
| Amber note | 5.1 | 4.0 | 4.2 | 3.9 | 4.6 |

From the results in Table 3, the flavor/fragrance composition (the flavor/fragrance composition of Example 2) using the compound represented by formula (1) of the present invention had an odor of a very clear and strong woody note which was more preferable than those of the flavor/fragrance compositions (Comparative Example 2 to 4) using ISO E SUPER, VERTFIX, and CASHMERAN, respectively, which are conventionally used compounds having an odor of a woody note, and achieved the highest score thereamong. In addition, the compound of the present invention was so excellent that the volume and diffusibility thereof were high.

### Example 3

### Preparation of Shampoo

A shampoo was prepared by using the flavor/fragrance composition prepared in Example 2 according to the formulation shown in Table 4 below. Specifically, the ratio of the compound represented by formula (1) mixed in the prepared shampoo was approximately 0.0025% by mass. The obtained shampoo favorably had an odor of a very clear and strong woody note.

TABLE 4

| | Parts by mass |
|---|---|
| Sodium polyoxyethylene lauryl ether sulfate | 14.00 |
| Lauramidopropyl betaine | 4.00 |
| Coconut oil fatty acid diethanolamide | 3.00 |
| Cationized cellulose | 0.50 |
| Ethylene glycol distearate | 1.00 |
| Paraoxybenzoic acid ester | 0.25 |
| Citric acid | Quantum Sufficit |
| Flavor/fragrance composition of Example 2 | 0.50 |
| Purified water | Balance |
| Total amount | 100.0 |

### Example 4

### Preparation of Hair Conditioner

A hair conditioner was prepared by using the flavor/fragrance composition prepared in Example 2 according to the formulation shown in Table 5 below. Specifically, the ratio of the compound represented by formula (1) mixed in the prepared hair conditioner was approximately 0.0025% by mass. The obtained hair conditioner favorably had an odor of a very clear and strong woody note.

TABLE 5

| | Parts by mass |
|---|---|
| Stearyltrimethylammonium chloride | 0.05 |
| Distearyl dimethylammonium chloride | 1.50 |
| Jojoba oil | 2.50 |
| Cetanol | 4.50 |
| Liquid lanolin | 2.00 |
| Polyoxyethylene stearyl alcohol ether | 1.50 |
| Concentrated glycerin | 7.00 |
| Paraoxybenzoic acid ester | 0.25 |
| Sodium hydroxide | Quantum Sufficit |
| Citric acid | Quantum Sufficit |
| Flavor/fragrance composition of Example 2 | 0.50 |
| Purified water | Balance |
| Total amount | 100.0 |

### Example 5

### Preparation of Body Shampoo

A body shampoo was prepared by using the flavor/fragrance composition prepared in Example 2 according to the formulation shown in Table 6 below. Specifically, the ratio of the compound represented by formula (1) mixed in the prepared body shampoo was approximately 0.0025% by mass. The obtained body shampoo favorably had an odor of a very clear and strong woody note.

TABLE 6

| | Parts by mass |
|---|---|
| Triethanolamine | 9.00 |
| Lauric acid | 6.00 |
| Myristic acid | 9.00 |
| Disodium lauryl polyoxyethylene sulfosuccinate | 10.00 |
| Alkyl(C8-C16)glycosides | 8.00 |
| 2-Hydroxyethyl distearate | 2.50 |
| Coconut oil fatty acid diethanolamide | 3.00 |
| Propylene glycol | 0.10 |
| Dibutylhydroxytoluene | 0.05 |
| Disodium edetate | 0.10 |
| Paraoxybenzoic acid ester | 0.25 |
| Flavor/fragrance composition of Example 2 | 0.50 |
| Purified water | Balance |
| Total amount | 100.0 |

### Example 6

### Preparation of Detergent Powder

A detergent powder was prepared by using the flavor/fragrance composition prepared in Example 2 according to the formulation shown in Table 7 below. Specifically, the ratio of the compound represented by formula (1) mixed in the prepared detergent powder was approximately 0.0029% by mass. The obtained detergent powder favorably had an odor of a very clear and strong woody note.

TABLE 7

| | Parts by mass |
|---|---|
| Sodium alkylbenzenesulfonate | 20 |
| Soap | 1 |
| Zeolite | 12 |
| Polycarboxylate | 1 |
| Soda ash | 20 |
| Salt cake | 30 |
| Fluorescent agent | 0.1 |
| Flavor/fragrance composition of Example 2 | 0.5 |
| Total amount | 84.6 |

### Example 7

### Preparation of Finishing Softener for Clothes

A finishing softener for clothes was prepared by using the flavor/fragrance composition prepared in Example 2 according to the formulation shown in Table 8 below. Specifically, the ratio of the compound represented by formula (1) mixed in the prepared finishing softener for clothes was approximately 0.0025% by mass. The obtained finishing softener for clothes favorably had an odor of a very clear and strong woody note.

TABLE 8

| | Parts by mass |
|---|---|
| Ethylene glycol | 6.0 |
| Polyoxyethylene oleyl cetyl ether | 2.0 |
| Dibutylhydroxytoluene | 0.1 |
| Anhydrous sodium sulfate | 0.8 |
| Imidazoline-type cationic surfactant CATION | 20.0 |
| SF-75PA (Sanyo Chemical Industries, Ltd.) | |
| Flavor/fragrance composition of Example 2 | 0.5 |
| Purified water | Balance |
| Total amount | 100.0 |

### Example 8

### Preparation of Hand Cream

A hand cream was prepared by using the flavor/fragrance composition prepared in Example 2 according to the formulation shown in Table 9 below. Specifically, the ratio of the compound represented by formula (1) mixed in the prepared hand cream was approximately 0.00025% by mass. The obtained hand cream favorably had an odor of a very clear and strong woody note.

TABLE 9

| | Parts by mass |
|---|---|
| Hydrogenated oil | 6.00 |
| Stearic acid | 3.00 |
| Cetanol | 4.00 |
| Squalane | 2.00 |
| Neopentyl glycol dicaprate | 8.00 |
| Polyoxyethylene sorbitan monostearate (20EO) | 4.00 |
| Lipophilic glycerin monostearate | 2.30 |
| N-Stearoyl-N-methyltaurine sodium salt | 1.70 |
| 1,3-Butylene glycol | 7.00 |
| Concentrated glycerin | 7.00 |
| Paraoxybenzoic acid ester | 0.25 |
| Flavor/fragrance composition of Example 2 | 0.05 |
| Purified water | Balance |
| Total amount | 100.0 |

### Example 9

### Preparation of Hair Tonic

A hair tonic was prepared by using the flavor/fragrance composition prepared in Example 2 according to the formulation shown in Table 10 below. Specifically, the ratio of the compound represented by formula (1) mixed in the prepared hair tonic was approximately 0.001% by mass. The obtained hair tonic favorably had an odor of a very clear and strong woody note.

TABLE 10

| | Parts by mass |
|---|---|
| Swertia herb extraction liquid | 2.00 |
| L-Menthol | 0.10 |
| Hinokitiol | 0.01 |
| Polyoxyethylene hydrogenated castor oil | 0.50 |
| Paraoxybenzoic acid ester | 0.25 |
| Flavor/fragrance composition of Example 2 | 0.20 |
| Purified water | Balance |
| Total amount | 100.0 |

### Example 10

### Preparation of Air-Freshening Mist

An air-freshening mist was prepared by using the flavor/fragrance composition prepared in Example 2 according to the formulation shown in Table 11 below. Specifically, the ratio of the compound represented by formula (1) mixed in the prepared air-freshening mist was approximately 0.0025% by mass. The obtained air-freshening mist favorably had an odor of a very clear and strong woody note.

TABLE 11

| | Parts by mass |
|---|---|
| Polyoxyethylene hydrogenated castor oil (EO40) | 1.00 |
| Polyoxyethylene hydrogenated castor oil (EO60) | 0.50 |
| 95% Ethyl alcohol | 2.50 |
| Methyl paraben | 0.10 |
| Flavor/fragrance composition of Example 2 | 0.50 |
| Purified water | Balance |
| Total amount | 100.0 |

### Example 11

### Preparation of Candle

A candle was prepared by using the flavor/fragrance composition prepared in Example 2 according to the formulation shown in Table 12 below. Specifically, the ratio of the compound represented by formula (1) mixed in the prepared candle was approximately 0.05% by mass. The obtained candle favorably had an odor of a very clear and strong woody note.

TABLE 12

| | Parts by mass |
|---|---|
| Paraffin wax | 22 |
| Stearic acid Pristerene | 20 |
| Beeswax, shea wax | 8 |
| White vaseline | 16 |
| Liquid paraffin | 24 |
| Flavor/fragrance composition of Example 2 | 10 |
| Total | 100 |

### Example 12

### Preparation of Air-Freshening Water-Based Gel

An air-freshening water-based gel was prepared by using the flavor/fragrance composition prepared in Example 2 according to the formulation shown in Table 13 below. Specifically, the ratio of the compound represented by formula (1) mixed in the prepared air-freshening water-based gel was approximately 0.025% by mass. The obtained air-freshening water-based gel favorably had an odor of a very clear and strong woody note.

TABLE 13

| | Parts by mass |
|---|---|
| Carrageenan | 2.00 |
| Locust bean gum | 0.40 |
| Methyl paraben | 0.10 |
| Calcium chloride | 0.40 |
| Propylene glycol | 3.00 |
| Polyoxyethylene sorbitan monooleate | 0.50 |
| Flavor/fragrance composition of Example 2 | 5.00 |
| Purified water | Balance |
| Total amount | 100.0 |

### Example 13

### Preparation of Air-Freshening Oil-Based Gel

An air-freshening oil-based gel was prepared by using the flavor/fragrance composition prepared in Example 2 according to the formulation shown in Table 14 below. Specifically, the ratio of the compound represented by formula (1) mixed in the prepared air-freshening oil-based gel was approximately 0.05% by mass. The obtained air-freshening oil-based gel favorably had an odor of a very clear and strong woody note.

TABLE 14

| | Parts by mass |
|---|---|
| Sodium stearate | 7.5 |
| Hexyleneglycol | 4.0 |
| Dibutylhydroxytoluene | 0.2 |
| Flavor/fragrance composition of Example 2 | 10.0 |
| Limonene | Balance |
| Total amount | 100.0 |

### Example 14

### Preparation of Flavor/Fragrance Composition

A flavor/fragrance composition was prepared by using the compound represented by formula (1) obtained in Example 1 according to the formulation shown in Table 15 below. Specifically, the ratio of the compound represented by formula (1) mixed in the prepared flavor/fragrance composition was approximately 0.48% by mass.

TABLE 15

| | Parts by mass |
|---|---|
| Ethyl acetate | 11 |
| cis-3-Hexenol | 3 |
| Hexanol | 5 |
| Benzaldehyde | 2 |
| Ethyl hexanoate | 22 |
| cis-3-Hexenyl acetate | 2 |
| Linalool | 70 |
| Linalool oxide | 4 |
| Hexyl butyrate | 25 |
| Hexyl hexanoate | 15 |
| α-Terpineol | 5 |
| Citral | 15 |
| cis-3-Hexenyl butyrate | 4 |
| cis-3-Hexenyl hexanoate | 5 |
| β-Ionone | 6 |
| Ethyl butyrate | 10 |
| 8-Mercaptomenthone | 0.01 |
| Compound of Example 1 | 1 |
| 95% Ethanol | Balance |
| Total amount | 210 |

### Example 15

### Preparation of Carbonated Beverage

A carbonated beverage (Brix: 9.3, acidity: 0.13% (in terms of citric acid), pH: 3.4, gas volume: 3.0) was prepared by using the flavor/fragrance composition prepared in Example 14 according to the formulation shown in Table 16 below. Specifically, the ratio of the compound represented by formula (1) mixed in the prepared carbonated beverage was approximately 0.0000873% by mass. The obtained carbonated beverage favorably had an odor of a very clear and strong woody note, and gave a natural feel.

TABLE 16

| | Parts by mass |
|---|---|
| High fructose corn syrup | 93.9 |
| Granulated sugar | 20.0 |
| Anhydrous citric acid | 1.30 |
| Sodium citrate | 0.10 |
| Ethanolic solution of 20% by mass flavor/ | 1.00 |
| fragrance composition prepared in Example 14 | |
| Water | 200 |
| Carbonated water | Balance |
| Total amount | 1100.0 |

### Example 16

### Preparation of Flavor/Fragrance Composition

A flavor/fragrance composition was prepared by using the compound represented by formula (1) obtained in Example 1 according to the formulation shown in Table 17 below. Specifically, the ratio of the compound represented by formula (1) mixed in the prepared flavor/fragrance composition was approximately 9.1% by mass.

TABLE 17

| | Parts by mass |
|---|---|
| Peppermint oil | 20 |
| l-Menthol | 40 |
| Spearmint oil | 10 |
| l-Carvone | 23 |
| Anethole | 6 |
| Lemon oil | 1 |
| Compound obtained in Example 1 | 10 |
| Total amount | 110 |

### Example 17

### Preparation of Tooth Cleaning Agent

A tooth cleaning agent was prepared by using the flavor/fragrance composition prepared in Example 16 according to the formulation shown in Table 18 below. Specifically, the ratio of the compound represented by formula (1) mixed in the prepared tooth cleaning agent was approximately 0.0091% by mass. The obtained tooth paste favorably had an odor of a very clear and strong woody note, and gave a natural feel.

TABLE 18

| | Parts by mass |
|---|---|
| Calcium hydrogen phosphate | 51.0 |
| Glycerin | 26.0 |
| Sodium lauryl sulfate | 1.4 |
| Carboxymethyl cellulose sodium | 1.0 |
| Sodium saccharin | 0.15 |
| Sodium benzoate | 0.05 |
| Ethanolic solution of 10% by mass flavor/ | 1.00 |
| fragrance composition prepared in Example 16 | |
| Purified water | Balance |
| Total amount | 100.0 |

The embodiments and examples disclosed herein are to be considered in all respects as illustrative and not restrictive. The scope of the present invention is indicated not by the above description, but by the scope of the claims, and the scope of the present invention is intended to include all modifications having an equivalent meaning to the scope of the claims and falling within the scope of the claims.

### INDUSTRIAL APPLICABILITY

When used alone as it is or in the form of a flavor/fragrance composition, the compound of the present invention makes it possible to efficiently provide an odor or flavor preferable for human beings to various products such as perfumes or cosmetics, foods or beverages, toiletry products, oral cavity compositions, and pharmaceuticals.

1. A compound represented by the following formula (1):

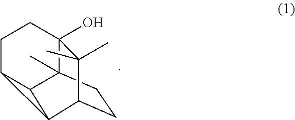

2. The compound according to claim 1, which is a (+)-isomer.

3. The compound according to claim 1, which is a (−)-isomer.

4. The compound according to claim 1, which is a mixture of a (+)-isomer and a (−)-isomer.

5. The compound according to claim 1, which is a racemate.

6. A method for producing a flavor/fragrance composition, comprising mixing the compound according to claim 1 with a flavor/fragrance component.

7. A method for producing a flavor/fragrance composition, comprising mixing the compound according to claim 2 with a flavor/fragrance component.

8. A method for producing a flavor/fragrance composition, comprising mixing the compound according to claim 3 with a flavor/fragrance component.

9. A method for producing a flavor/fragrance composition, comprising mixing the compound according to claim 4 with a flavor/fragrance component.

10. A method for producing a flavor/fragrance composition, comprising mixing the compound according to claim 5 with a flavor/fragrance component.

11. A method for producing a perfume, a cosmetic, a toiletry product, a food, a beverage, an oral cavity composition, or a pharmaceutical, the method comprising:
mixing the compound according to claim 1 with a flavor/fragrance component, to thereby obtain a flavor/fragrance composition; and
incorporating the obtained flavor/fragrance composition into a perfume, a cosmetic, a toiletry product, a food, a beverage, an oral cavity composition, or a pharmaceutical.

12. A method for producing a perfume, a cosmetic, a toiletry product, a food, a beverage, an oral cavity composition, or a pharmaceutical, the method comprising:
mixing the compound according to claim 2 with a flavor/fragrance component, to thereby obtain a flavor/fragrance composition; and
incorporating the obtained flavor/fragrance composition into a perfume, a cosmetic, a toiletry product, a food, a beverage, an oral cavity composition, or a pharmaceutical.

13. A method for producing a perfume, a cosmetic, a toiletry product, a food, a beverage, an oral cavity composition, or a pharmaceutical, the method comprising:
mixing the compound according to claim 3 with a flavor/fragrance component, to thereby obtain a flavor/fragrance composition; and
incorporating the obtained flavor/fragrance composition into a perfume, a cosmetic, a toiletry product, a food, a beverage, an oral cavity composition, or a pharmaceutical.

14. A method for producing a perfume, a cosmetic, a toiletry product, a food, a beverage, an oral cavity composition, or a pharmaceutical, the method comprising:
mixing the compound according to claim 4 with a flavor/fragrance component, to thereby obtain a flavor/fragrance composition; and
incorporating the obtained flavor/fragrance composition into a perfume, a cosmetic, a toiletry product, a food, a beverage, an oral cavity composition, or a pharmaceutical.

15. A method for producing a perfume, a cosmetic, a toiletry product, a food, a beverage, an oral cavity composition, or a pharmaceutical, the method comprising:
mixing the compound according to claim 5 with a flavor/fragrance component, to thereby obtain a flavor/fragrance composition; and
incorporating the obtained flavor/fragrance composition into a perfume, a cosmetic, a toiletry product, a food, a beverage, an oral cavity composition, or a pharmaceutical.

16. A method for producing a perfume, a cosmetic, a toiletry product, a food, a beverage, an oral cavity composition, or a pharmaceutical, the method comprising incorporating the compound according to claim 1 into a perfume, a cosmetic, a toiletry product, a food, a beverage, an oral cavity composition, or a pharmaceutical.

17. A method for producing a perfume, a cosmetic, a toiletry product, a food, a beverage, an oral cavity composition, or a pharmaceutical, the method comprising incorporating the compound according to claim 2 into a perfume, a cosmetic, a toiletry product, a food, a beverage, an oral cavity composition, or a pharmaceutical.

18. A method for producing a perfume, a cosmetic, a toiletry product, a food, a beverage, an oral cavity composition, or a pharmaceutical, the method comprising incorporating the compound according to claim 3 into a perfume, a cosmetic, a toiletry product, a food, a beverage, an oral cavity composition, or a pharmaceutical.

19. A method for producing a perfume, a cosmetic, a toiletry product, a food, a beverage, an oral cavity composition, or a pharmaceutical, the method comprising incorporating the compound according to claim 4 into a perfume, a cosmetic, a toiletry product, a food, a beverage, an oral cavity composition, or a pharmaceutical.

20. A method for producing a perfume, a cosmetic, a toiletry product, a food, a beverage, an oral cavity composition, or a pharmaceutical, the method comprising incorporating the compound according to claim 5 into a perfume, a cosmetic, a toiletry product, a food, a beverage, an oral cavity composition, or a pharmaceutical.
